(19) United States
(12) Patent Application Publication
(10) Pub. No.: US 2021/0158900 A1
(43) Pub. Date: May 27, 2021

(54) A METHOD AND SYSTEM FOR GENE SIGNATURE MARKER SELECTION

(71) Applicant: KONINKLIJKE PHILIPS N.V., EINDHOVEN (NL)

(72) Inventors: Yee Him Cheung, Boston, MA (US); Nevenka Dimitrova, Pelham Manor, NY (US); Wilhelmus Franciscus Johannes Verhaegh, Eindhoven (NL)

(21) Appl. No.: 16/624,526

(22) PCT Filed: Jun. 25, 2018
(86) PCT No.: PCT/EP2018/067001

§ 371 Date: Dec. 19, 2019

Related U.S. Application Data
(60) Provisional application No. 62/524,112, filed on Jun. 23, 2017.

(51) Int. Cl. G16B 40/00; G16B 25/00; G06F 17/18; G16B 50/30

(52) U.S. Cl. CPC G16B 40/00; G16B 50/30; G06F 17/18; G16B 25/00

(57) ABSTRACT

A method (100) comprising: (i) providing (104, 106) a reference data set for a first gene expression platform and a training data set for a second platform; (iii) computing (108) a boundary expression level that distinguishes between the subtypes; (iv) generating (1 10) a confusion matrix; (v) determining (112) an effectiveness of the gene markers to discriminate between the subtypes; (vi) filtering (114) any gene marker with an expression level below a threshold or with determined effectiveness below a threshold; (v) calculating (118) a mean and/or median expression level for each subtype, and comparing them to generate an expression level change; (vi) comparing (118) the expression level change to a reference expression level change; and (vii) selecting (120) the gene marker if the generated expression level change and the reference expression level change are changed in the same direction; and (viii) providing (124) the selected gene markers via a user interface.

### CROSS-REFERENCE TO RELATED APPLICATIONS

This application claims priority to U.S. Provisional Patent Application Ser. No. 62/524,112, filed on Jun. 23, 2017, and entitled “A Method and System for Gene Signature Marker Selection,” the entire contents of which are incorporated herein by reference.

### FIELD OF THE DISCLOSURE

The present disclosure is directed generally to methods and systems for selecting gene markers.

### BACKGROUND

The dynamic range of gene expression can vary considerably depending on the choice of profiling platform and the type of tissue under study. As a result, prognostic gene signatures are usually platform- and/or tissue-specific. Gene signatures used to analyze a condition and/or provide predictive information typically comprise a plurality of gene markers. These gene markers are selected to enhance the classification performance of a gene signature for a specific platform by, for example, excluding the weak or non-discriminative markers whose measurements may present as noise and hence affect the overall performance of subtype classification.

Some gene markers, such as probes, exons, isoforms, or simply the genes themselves, that work effectively for the subtyping of samples on one type of analytical platform are weak or non-discriminative on a different analytical platform. These gene markers are therefore incompatible between the two analytical platforms.

For example, many gene signatures comprising a plurality of gene markers are designed primarily for microarray platforms. However, some of the gene markers selected for the gene signature are not suitably detectable, replicable, and/or discriminative on a different analytical platform such as RNA-Seq platforms. Accordingly, the gene signature designed for the microarray platform will not perform sufficiently for the RNA-Seq platform. This incompatibility limits the scope of use of a genetic signature comprising these markers, increases the cost of running multiple signature-based diagnostic tests on a patient, and can hinder the adoption of new profiling technologies, among other limitations.

### SUMMARY OF THE DISCLOSURE

There is a continued need for methods and systems for selecting gene markers that are compatible between two or more analytical platforms.

The present disclosure is directed to inventive methods and systems for selecting suitable gene markers from a plurality of gene marker candidates. Various embodiments and implementations herein are directed to a data-driven integrative method and system for optimizing gene expression signatures used on a specific primary profiling platform for subtype classification, such that the subtyping performance of the gene expression signature is enhanced for use on the primary platform or on alternative platforms. Specifically, the method utilizes a gene marker selection method that optimizes an expression signature by choosing a subset of markers that individually have sufficient detectability and discriminative power. The gene markers chosen could be probes, exons, isoforms or the gene itself. As an example, the marker selection method of this invention can be used to adapt a microarray-based gene signature for an RNA-Seq expression platform, although many other types of platforms may be utilized.

Generally in one aspect, is a method for identifying a gene signature comprising one or more selected gene markers each configured to discriminate between two or more tissue or cell subtypes is provided. The method includes: (i) identifying a set of candidate gene markers for discrimination of the two or more subtypes; (ii) providing a reference data set for a first gene expression platform, comprising expression information for a plurality of genes, and further comprising information about a difference in expression of at least some of the plurality of genes between a first subtype and a second subtype; (iii) providing a training data set for a second gene expression platform, comprising expression information for a plurality of genes for each of the first subtype and the second subtype, wherein at least some of the plurality of genes in the training data set are within the set of candidate gene markers; (iv) computing a boundary gene expression level that distinguishes between the first subtype and the second subtype; (v) generating, using the computed boundary gene expression level and the expression information for the plurality of genes in the training data set, a confusion matrix for the first subtype and the second subtype; (vi) determining, based on the generated confusion matrix, an effectiveness of each of the plurality of genes in the training data set to discriminate between the first subtype and the second subtype; (vii) filtering from further consideration any of the plurality of genes in the training data set with an expression level below a threshold; (viii) filtering from further consideration any of the plurality of genes in the training data set with a determined effectiveness below a threshold; (ix) calculating a mean and/or median expression level of one of the remaining plurality of genes in the training data set for the first subtype and for the second subtype, and comparing the two calculated mean and/or median expression levels to generate an expression level change for each gene marker between the first subtype and the second subtype; (x) comparing the generated expression level change for the gene marker to a reference expression level change for the same gene marker in the reference data set; and (xi) selecting the gene marker for inclusion in a list of selected gene markers if the generated expression level change and the reference expression level change are changed in the same direction; and (xii) providing the list of one or more selected gene markers via a user interface, the list comprising the gene signature.

In accordance with an embodiment, the method further includes: identifying a gene marker in the training data set for which the generated expression level change and the reference expression level change are changed in opposite directions; and selecting the gene marker for inclusion in the list of selected gene markers if the gene marker comprises an expression level above a threshold in the training data set, or if the gene marker comprises an expression level below a threshold in the reference data set.

In accordance with an embodiment, one or more of the thresholds is a user-selected threshold.

In accordance with an embodiment, the expression level threshold comprises a mean or median expression level obtained from expression information for the plurality of genes in the training data set.

In accordance with an embodiment, the effectiveness of each of the plurality of genes in the training data set is determined using one or more of sensitivity, specificity, a Matthew correlation coefficient, and hypergeometric p values.

In accordance with an embodiment, the effectiveness of each of the plurality of genes in the training data set is determined using a Matthew correlation coefficient (MCC) and the formula:

$$MCC = \frac{TP \times TN - FP \times FN}{\sqrt{( TP + FP ) \times ( TN + FN ) \times P \times N}}$$

where TP=true positives, FP=false positives, TN=true negatives, FN=false negatives, P=TP+FN, and N=PF+TN.

In accordance with an embodiment, the first subtype and the second subtype are cancer subtypes.

In accordance with an embodiment, the first gene expression platform is a microarray platform, and the second gene expression platform is an RNA platform.

Generally, in one aspect, a system for identifying a gene signature comprising one or more selected gene markers each configured to discriminate between two or more tissue or cell subtypes is provided. The system includes: a reference data set comprising expression information obtained from a first gene expression platform for a plurality of genes, and further comprising information about a difference in expression of at least some of the plurality of genes between a first subtype and a second subtype; a training data set comprising expression information obtained from a second gene expression platform for a plurality of genes, and further comprising information about a difference in expression of at least some of the plurality of genes between a first subtype and a second subtype; a processor configured to: (i) compute a boundary gene expression level that distinguishes between the first subtype and the second subtype; (ii) generate, using the computed boundary gene expression level and the expression information for the plurality of genes in the training data set, a confusion matrix for the first subtype and the second subtype; (iii) determine, based on the generated confusion matrix, an effectiveness of each of the plurality of genes in the training data set to discriminate between the first subtype and the second subtype; (iv) filter from further consideration any of the plurality of genes in the training data set with an expression level below a threshold; (v) filter from further consideration any of the plurality of genes in the training data set with a determined effectiveness below a threshold; (vi) calculate a mean and/or median expression level of one of the remaining plurality of genes in the training data set for the first subtype and for the second subtype, and comparing the two calculated mean and/or median expression levels to generate an expression level change for each gene marker between the first subtype and the second subtype; (viii) compare the generated expression level change for the gene marker to a reference expression level change for the same gene marker in the reference data set; and (ix) select the gene marker for inclusion in a list of selected gene markers if the generated expression level change and the reference expression level change are changed in the same direction; and further includes a user interface configured to provide the list of one or more selected gene markers via a user interface, the list comprising the gene signature.

In accordance with an embodiment, the processor is further configured to: identify a gene marker in the training data set for which the generated expression level change and the reference expression level change are changed in opposite directions; and select the gene marker for inclusion in the list of selected gene markers if the gene marker comprises an expression level above a threshold in the training data set, or if the gene marker comprises an expression level below a threshold in the reference data set.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for selecting gene markers, in accordance with an embodiment.

FIG. 2 is a confusion matrix utilized in a method for selecting gene markers, in accordance with an embodiment.

FIG. 3 is a schematic representation of a system for selecting gene markers, in accordance with an embodiment.

FIG. 4A is a histogram of log₂ expressions for an Affymetrix (single-channel) microarray platform, in accordance with an embodiment.

FIG. 4B is a histogram of log₂ expressions for an Agilent (two-channel) microarray platform, in accordance with an embodiment.

FIG. 4C is a histogram of log₂ expressions for an Illumina HiSeq 2000 RNA-Seq platform, in accordance with an embodiment.

FIG. 5 is a graph of a correlation and concordance of predicted Wnt pathway activity and status with and without a gene marker selection method, in accordance with an embodiment.

### DETAILED DESCRIPTION OF EMBODIMENTS

The present disclosure describes various embodiments of a system and method for selecting suitable gene markers from a plurality of gene marker candidates. More generally, Applicant has recognized and appreciated that it would be beneficial to provide a system configured to select gene markers that are compatible between two or more analytical platforms. The system identifies gene markers for the classification of subtypes from a set of candidate gene markers. The system computes a boundary gene expression level that distinguishes between a first subtype and a second subtype, and uses the boundary to generate a confusion matrix for the first subtype and the second subtype. The system uses the confusion matrix to determine the effectiveness of each candidate gene marker to discriminate between the first subtype and the sample subtype. Candidate gene markers with a determined effectiveness below a threshold, or with an expression level below a threshold, are filtered out from further consideration. The system selects candidate gene marker for which the difference between the expression level of each of the remaining candidate gene markers for the first subtype and the second subtype using the first analytical platform is in the same direction as difference using the second analytical platform. The system then displays or otherwise provides a list of one or more selected gene markers on a graphical user interface.

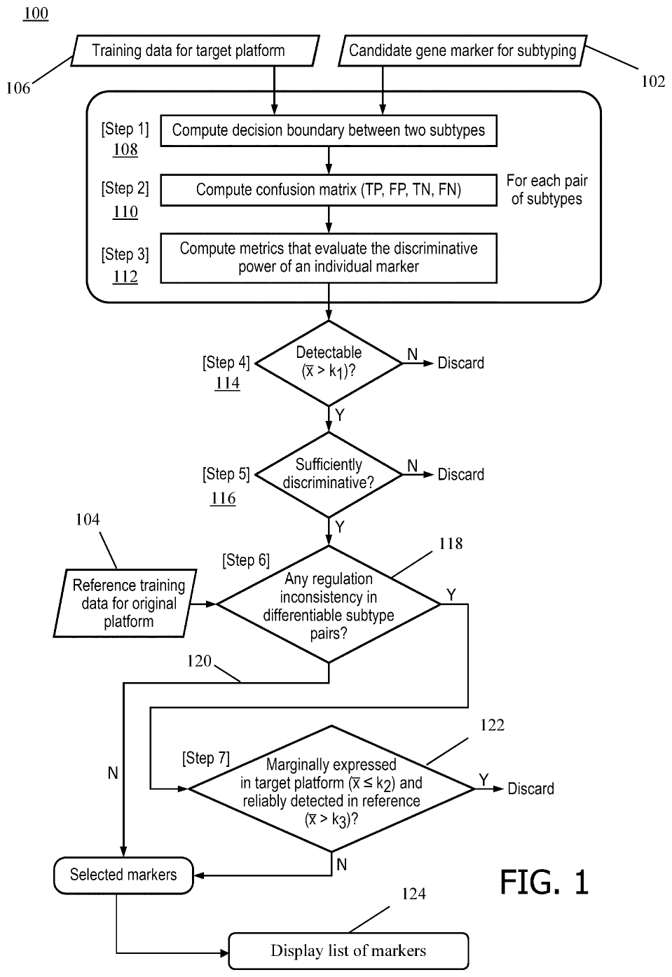

Referring to FIG. 1, in one embodiment, is a flowchart of a method 100 for selecting one or more gene markers using a gene marker selection system. The gene marker selection system may be any of the systems described or otherwise envisioned herein, and may comprise any of the components described or otherwise envisioned herein.

At step 102 of the method, a set of candidate gene markers for the classification of two or more subtypes are identified. The candidate gene markers may be any measurable marker, including but not limited to probes, exons, isoforms, or CNVs, genotypes, expression levels, and/or any other marker measures by one or more platform. Many other measurable markers are possible.

The subtypes may be any distinguishable cellular or tissue subtypes. For example, the subtypes may be cancer subtypes, or sub-classifications of a cancer based on one or more phenotypic or genotypic characterizations, such as cell shape or other physical characteristic, and/or genotype differences characterized by sequencing or other DNA or RNA analysis. Subtypes such as cancer subtypes may respond different to treatment and/or have different predictive capabilities.

At step 104 of the method, a reference data set from a first analytical platform is provided. The reference data set is utilized in a downstream step for comparison to one or more markers selected based on training data. The reference data set is obtained from, for example, a plurality of samples of the two or more subtypes. According to just one embodiment, a first primary analytical platform may be microarray analysis, and a second or secondary analytical platform may be RNA-Seq analysis, or vice versa, although many other analytical platforms may be utilized according to the methods and systems described or otherwise envisioned herein. Each of the samples in the reference data set are associated with a classification or other identification of the particular or most likely subtype from which the sample was obtained. The classification or identification may be based on the data from the first analytical platform, or may be based on other phenotypic and/or genotypic analysis. Therefore, according to an example, the reference data set may be microarray data obtained from a plurality of samples of each of two or more cancer subtypes, in which the microarray data for each sample is associated with the specific cancer subtype.

At step 106 of the method, a training data set from a secondary analytical platform is provided. The training data is utilized to generate a model for the gene marker selection system, and comprises a reference data set for a first gene expression analysis platform. The reference data set comprises, for example, expression information for a plurality of gene markers for each of the two or more subtypes. The reference data is obtained from, for example, a plurality of samples of each of the two or more subtypes. According to just one embodiment, the second or secondary analytical platform may be RNA-Seq analysis, although many other analytical platforms may be utilized according to the methods and systems described or otherwise envisioned herein.

The training data set may be provided to the gene marker selection system from any local or remote source. For example, the training data set may be provided to the gene marker selection system in real time, or may be stored in a database and provided to the gene marker system via any data transfer method.

At step 108 of the method, the gene marker selection system computes a boundary gene expression level that distinguishes between two of the two or more subtypes. The target-specific decision boundary gene expression level between two subtypes can be computed from the training data in many ways. One approach is a weighted median, where each sample belonging to a subtype is assigned the same weight such that the total weights of the two groups are equal. The median is then found by identifying the point that corresponds to half of the total weight. Another approach is to assume that the two subtypes follow normal distributions with different means and standard deviation. The boundary is then set as the intersection of the two distributions, approximated by averaging the two groups means weighted by the inverse of their respective standard deviations. A third approach is to find a boundary that optimizes the discriminative power of the training data as measured by a method such as Matthew correlation coefficient (MCC), among others. According to an embodiment, the boundary can be set as the average of the means of the two subtypes.

At step 110 of the method, the gene marker selection system generates, using the computed boundary gene expression level, a confusion matrix for the first sample subtype and the second sample subtype. The confusion matrix is, for example, a matrix in which each row represents instances of training data in a predicted subtype while each column represents instances in an actual subtype (or vice versa). The confusion matrix may be utilized to describe or test the performance of the gene marker selection system, and/or a classification model or classifier of the system, to classify the training data.

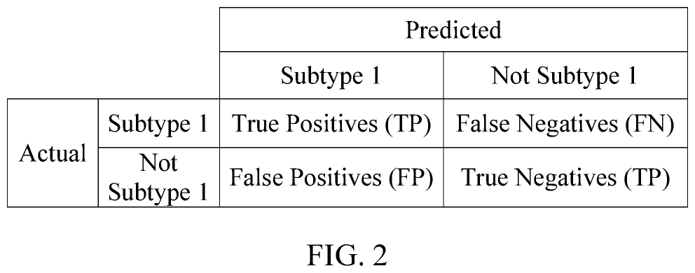

Referring to FIG. 2, in one embodiment, is an example of a confusion matrix in which each row represents instances of training data in an actual subtype (i.e., Subtype 1 or Not Subtype 1), while each column represents instances in a predicted subtype (i.e., Subtype 1 or Not Subtype 1). The data is used to populate the cells, corresponding to true positives (TP), false positives (FP), true negatives (TN) and false negatives (FN), with classification based on the boundary gene expression level obtained in step 108 of the method.

The confusion matrix may be computed using any of a different number of methods. According to an embodiment, one approach for computing the confusion matrix is hard counting. This method weighs the expression levels of all samples equally regardless of their relative distances from the decision boundary. According to another embodiment, another approach for computing the confusion matrix is soft counting in which it is assumed that all the expression of a gene marker follows a Gaussian normal distribution centered at the boundary, with the standard deviation computed from the observed measurements. Based on that distribution, the system can estimate for each measurement a probability of being associated with one subtype. The entries of the confusion matrix can then be found by properly summing these probabilities. Many other methods for generating the confusion matrix are possible.

At step 112 of the method, the gene marker selection system determines, using the generated confusion matrix, an effectiveness of some or all of the candidate gene markers to discriminate between the two subtypes. The gene marker selection system may use any of a variety of different methods to determine the effectiveness of a gene marker in the training data set to discriminate between the two subtypes. According to an embodiment, some of the possible mechanisms or metrics include sensitivity, specificity, Matthew correlation coefficient (MCC; 1 for total agreement, −1 for total disagreement), and/or hypergeometric p values, among many others. The definitions of some of the metrics are as follows:

$$\begin{matrix}P = TP + FN & ( Eq . 1 ) \\ N = PF + TN & ( Eq . 2 ) \\ Sensitivity = TP / P & ( Eq . 3 ) \\ Specificity = TN / N & ( Eq . 4 ) \\ MCC = \frac{TP \times TN - FP \times FN}{\sqrt{( TP + FP ) \times ( TN + FN ) \times P \times N}} & ( Eq . 5 )\end{matrix}$$

Among other possibilities, a candidate gene marker may be ineffective at discriminating between any subtypes, a candidate gene marker may be effective at discriminating between only two subtypes, or a candidate gene marker may be effective at discriminating between more than two subtypes.

According to an embodiment, each of the candidate gene markers will be tagged, flagged, marked, associated, or otherwise indicated as being effective or ineffective, optionally along with a measurement of that effectiveness or ineffectiveness, at discriminating between two or more subtypes. For example, the indication may be an association between the candidate gene marker and each of the two or more subtypes in memory, optionally with additional information about a measurement of effectiveness or ineffectiveness, or may be stored in a data structure such as a table, among many other options.

Steps 108 through 112 can be repeated for each pair of subtypes among the two or more subtypes. For example, if there are three (3) subtypes called Subtype A, Subtype B, and Subtype C, steps 108 through 112 can be repeated for the following combinations: (1) Subtype A and Subtype B; (2) Subtype A and Subtype C; and (3) Subtype B and Subtype C. A gene biomarker will then be selected if it passes the filtering steps for at least one or multiple of these combinations depending on the user's criteria.

At step 112 of the method, the gene marker selection system filters out from further consideration, or otherwise discards, categorizes, and/or flags, any of the candidate gene markers that comprise an expression level below a threshold. Similarly, the gene marker selection system can mark for further consideration, or otherwise retain, categorize, and/or flag, any of the candidate gene markers that comprise an expression level above a threshold. This ensures that data from a candidate gene marker will be detectable and accurate, and will therefore be useful in this and other analyses, as low expressions may be more prone to measurement errors.

The threshold can be any threshold, including a user-defined or user-selected threshold, a system-defined or system-selected threshold, an experimentally derived threshold, and/or any other threshold. Accordingly, the gene marker selection system may comprise a setting, user interface, or other interaction mechanism whereby a user can set or select a threshold. According to another embodiment, the gene marker selection system may determine a suitable threshold based on all or a portion of the gene expression information in the training data set data. According to an embodiment, the expression level is determined using a mean or median expression level obtained from the plurality of samples in the training data, such as X̅>k₁, among other methods.

At step 114 of the method, the gene marker selection system filters out from further consideration, or otherwise discards, categorizes, and/or flags, any of the candidate gene markers that comprise a determined effectiveness below a threshold. Similarly, the gene marker selection system can mark for further consideration, or otherwise retain, categorize, and/or flag, any of the candidate gene markers that comprise an effectiveness above a threshold. This effectiveness determination or characterization is obtained from, for example, step 112 of the method. This ensures that the data from a candidate gene marker will provide relevant information to discriminate between any two of the two or more subtypes, and at a sufficiently discriminative level. Any method for determining effectiveness may be utilized, including but limited to the methods described in conjunction to step 112, including equations 1-5.

The threshold can be any threshold, including a user-defined or user-selected threshold, a system-defined or system-selected threshold, an experimentally derived threshold, and/or any other threshold. Accordingly, the gene marker selection system may comprise a setting, user interface, or other interaction mechanism whereby a user can set or select a threshold. According to another embodiment, the gene marker selection system may determine a suitable threshold based on all or a portion of the data in the training data set data.

If a gene signature works robustly on a first platform, and the goal is to adapt it for a second platform, given that training data is also available for the first platform, then one or more additional steps may be applied to identify and resolve any cross-platform discrepancies in the regulation of expression between subtypes. Note that if a marker is not directly available in the first platform, it may instead be compared with the corresponding gene-level expression.

At step 118 of the method, the gene marker selection system determines a mean or median expression level of a gene marker that has survived filtering in steps 114 and 116 and remains a candidate gene marker, for each of two or more subtypes for which that gene marker is discriminative. The system compares the mean or median expression level of the gene marker for the first subtype to the mean or median expression level of the same gene marker for the second subtype, to generate an expression level change. Since the gene marker has survived filtering and remains a candidate gene marker, the expression level difference is detectable and effective for discriminating between the two subtypes. This expression level change will either be a positive change in expression or a negative change in expression, depending on the characteristics of the gene marker in the first and second subtypes. The result of this portion of step 118 is a calculated positive or negative expression level difference in the training data set, such as in the form of a quantified value, for the gene marker candidate.

Also at step 118 of the method, the gene marker selection system similarly determines a mean or median expression level of the same gene marker from the reference data set provided in step 104 of the method. Alternatively, the gene marker selection system receives this mean or median expression level from an external source. According to an embodiment, the reference data set comprises or is used to determine or calculate the mean or median expression level of the same gene marker for each of two or more subtypes used in step 116. The system compares the mean or median expression level of the gene marker for the first subtype to the mean or median expression level of the same gene marker for the second subtype, to generate an expression level change. This expression level change will either be a positive change in expression or a negative change in expression, depending on the characteristics of the gene marker in the first and second subtypes. The result of this portion of step 118 is a calculated positive or negative expression level difference in the reference data set, such as in the form of a quantified value, for the gene marker candidate.

Accordingly, the gene marker selection system comprises, for this gene marker, a first expression level difference obtained from the training data set and the second analytical platform, and a second expression level difference obtained from the reference data set and the first analytical platform.

Also at step 118 of the method, the gene marker selection system compares the first expression level difference obtained from the training data set and the second analytical platform to the second expression level difference obtained from the reference data set and the first analytical platform.

At step 120 of the method, if the first expression level difference and the second expression level difference change in the same direction, such as if the first expression level difference is a positive difference and the second expression level difference is also a positive difference, then the candidate gene marker is selected for inclusion in the final list of selected gene markers. Similarly, if the first expression level difference is a negative difference and the second expression level difference is also a negative difference, then the candidate gene marker is selected for inclusion in the final list of selected gene markers. According to an embodiment, if the first expression level difference and the second expression level difference change in opposite directions, the candidate gene marker is filtered out from further consideration, or otherwise discarded, categorized, and/or flagged.

According to an embodiment, the steps of step 118 and step 120 are repeated for each gene marker that has survived filtering in steps 114 and 116 and remains a candidate gene marker, thereby creating a completed final list of selected gene markers.

If the first expression level difference and the second expression level difference change in opposite directions, the candidate gene marker may be filtered out from further consideration, or otherwise discarded, categorized, and/or flagged. However, according to an embodiment, candidate gene markers for which the first expression level difference and the second expression level difference change in opposite directions can be further analyzed. In this way, a gene marker from the training data set obtained from the second analytical platform may optionally still be included in the list of selected gene markers if it has survived filtering in steps 114 and 116 and thus is detectable and discriminative.

Accordingly, at step 122 of the method, these gene markers may be selected for inclusion in the final list of selected gene markers if: (1) the candidate gene marker comprises an expression level above a threshold in the training data set obtained from the second analytical platform; and/or (2) the candidate gene marker comprises an expression level below a threshold in the reference data set obtained from the first analytical platform. For example, according to embodiment, the candidate gene marker is included if the marker is reliably detected in the alternative platform, i.e. x̅>k₂, or negligibly expressed in the primary platform, i.e. x̅′≤k₃. Parameters k₁, k₂ and k₃ can be adjusted according to the platforms, with k₂>k₁ in general, according to an embodiment.

At step 124 of the method, the final list of selected gene markers is displayed to a user on a graphical user interface, or otherwise provided to a user. For example, the list may be displayed on a screen, provided in a data output such as a text file or a spreadsheet, printed, or otherwise provided. Thus, according to an embodiment, the gene marker selection system comprises or is in communication with a graphical user interface or other device to provide the final list of selected gene markers to the user.

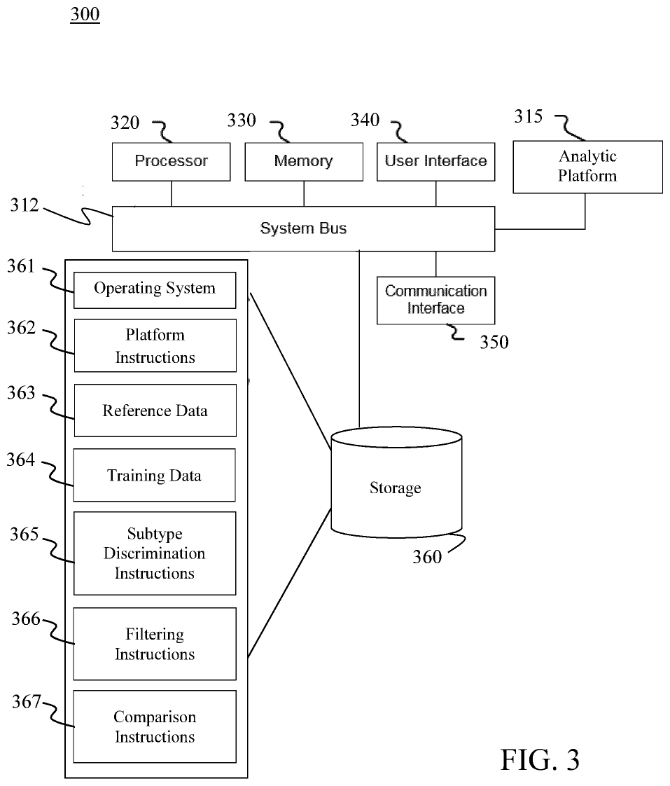

Referring to FIG. 3, in one embodiment, is a schematic representation of a gene marker selection system 300 for generating a list of selected gene markers for one or more gene signatures. System 300 may be any of the systems described or otherwise envisioned herein, and may comprise any of the components described or otherwise envisioned herein.

According to an embodiment, system 300 comprises one or more of a processor 320, memory 330, user interface 340, communications interface 350, and storage 360, interconnected via one or more system buses 312. In some embodiments, such as those where the system comprises or directly implements an analytic platform 315, the hardware may include additional analytic hardware such as microarray and/or RNA-Seq hardware, although many other sequencing platforms are possible. It will be understood that FIG. 3 constitutes, in some respects, an abstraction and that the actual organization of the components of the system 300 may be different and more complex than illustrated.

According to an embodiment, system 300 comprises a processor 320 capable of executing instructions stored in memory 330 or storage 360 or otherwise processing data to, for example, perform one or more steps of the method. Processor 320 may be formed of one or multiple components. Processor 320 may take any suitable form, including but not limited to a microprocessor, microcontroller, multiple microcontrollers, circuitry, field programmable gate array (FPGA), application-specific integrated circuit (ASIC), a single processor, or plural processors.

Memory 330 can take any suitable form, including a non-volatile memory and/or RAM. The memory 330 may include various memories such as, for example L1, L2, or L3 cache or system memory. As such, the memory 330 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 300. It will be apparent that, in embodiments where the processor implements one or more of the functions described herein in hardware, the software described as corresponding to such functionality in other embodiments may be omitted.

User interface 340 may include one or more devices for enabling communication with a user. The user interface can be any device or system that allows information to be conveyed and/or received, and may include a display, a mouse, and/or a keyboard for receiving user commands. In some embodiments, user interface 340 may include a command line interface or graphical user interface that may be presented to a remote terminal via communication interface 350. The user interface may be located with one or more other components of the system, or may located remote from the system and in communication via a wired and/or wireless communications network. User interface 340 may comprise, for example, a screen for display of the list of selected gene markers for one or more gene signatures. User interface 340 may comprise, for example, a data output or retrieval mechanism for obtaining or downloading the list of selected gene markers for one or more gene signatures. According to an embodiment, user interface 340 may display the final list of selected gene markers to a user, or otherwise provide the list to a user. For example, the list may be displayed on a screen, provided in a data output such as a text file or a spreadsheet, printed, or otherwise provided.

Communication interface 350 may include one or more devices for enabling communication with other hardware devices. For example, communication interface 350 may include a network interface card (NIC) configured to communicate according to the Ethernet protocol. Additionally, communication interface 350 may implement a TCP/IP stack for communication according to the TCP/IP protocols. Various alternative or additional hardware or configurations for communication interface 350 will be apparent.

Storage 360 may include one or more machine-readable storage media such as read-only memory (ROM), random-access memory (RAM), magnetic disk storage media, optical storage media, flash-memory devices, or similar storage media. In various embodiments, storage 360 may store instructions for execution by processor 320 or data upon which processor 320 may operate. For example, storage 360 may store an operating system 361 for controlling various operations of system 300. Where system 300 implements an analytic platform 315 such as a microarray or RNA-Seq platform, storage 360 may include instructions 362 for operating the analytic platform 315.

It will be apparent that various information described as stored in storage 360 may be additionally or alternatively stored in memory 330. In this respect, memory 330 may also be considered to constitute a storage device and storage 360 may be considered a memory. Various other arrangements will be apparent. Further, memory 330 and storage 360 may both be considered to be non-transitory machine-readable media. As used herein, the term non-transitory will be understood to exclude transitory signals but to include all forms of storage, including both volatile and non-volatile memories.

According to an embodiment, system 300 comprises or is in communication with a reference data set 363 obtained from a first analytical platform. For example, storage 360 may comprise the reference data set 363. The reference data set is obtained from, for example, a plurality of samples of the two or more subtypes. According to just one embodiment, a first primary analytical platform may be microarray analysis, and a second or secondary analytical platform may be RNA-Seq analysis, or vice versa, although many other analytical platforms may be utilized according to the methods and systems described or otherwise envisioned herein. Each of the samples in the reference data set are associated with a classification or other identification of the particular or most likely subtype from which the sample was obtained. The classification or identification may be based on the data from the first analytical platform, or may be based on other phenotypic and/or genotypic analysis. Therefore, according to an example, the reference data set may be microarray data obtained from a plurality of samples of each of two or more cancer subtypes, in which the microarray data for each sample is associated with the specific cancer subtype.

According to an embodiment, system 300 comprises or is in communication with a training data set 364 obtained from a secondary analytical platform. For example, storage 360 may comprise the training data set 364. The reference data set comprises, for example, expression information for a plurality of gene markers for each of the two or more subtypes. The reference data is obtained from, for example, a plurality of samples of each of the two or more subtypes. According to just one embodiment, the second or secondary analytical platform may be RNA-Seq analysis, although many other analytical platforms may be utilized according to the methods and systems described or otherwise envisioned herein.

According to an embodiment, storage 360 of gene marker selection system 300 may store one or more algorithms and/or instructions to carry out one or more functions or steps of the methods described or otherwise envisioned herein. For example, processor 320 may comprise subtype discrimination instructions 365, filtering instructions 366, and/or comparison instructions 367.

According to an embodiment, subtype discrimination instructions 365 direct the system to compute a boundary gene expression level that distinguishes between two of the two or more subtypes. The target-specific decision boundary gene expression level between two subtypes can be computed from the training data in many ways, including the methods described or otherwise envisioned herein.

According to an embodiment, subtype discrimination instructions 365 direct the system to generate, using the computed boundary gene expression level, a confusion matrix for the first sample subtype and the second sample subtype. The confusion matrix may be utilized to describe or test the performance of the gene marker selection system, and/or a classification model or classifier of the system, to classify the training data. The confusion matrix may be computed using any of a different number of methods, including the methods described or otherwise envisioned herein.

According to an embodiment, subtype discrimination instructions 365 direct the system to determine, using the generated confusion matrix, an effectiveness of some or all of the candidate gene markers to discriminate between the two subtypes. The gene marker selection system may use any of a variety of different methods to determine the effectiveness of a gene marker in the training data set to discriminate between the two subtypes, including the methods described or otherwise envisioned herein.

According to an embodiment, filtering instructions 366 direct the system to filter out from further consideration any of the candidate gene markers that comprise an expression level below a threshold. The candidate gene markers may alternatively be discarded, categorized, and/or flagged for falling below the threshold. The threshold can be any threshold, including a user-defined or user-selected threshold, a system-defined or system-selected threshold, an experimentally derived threshold, and/or any other threshold.

According to an embodiment, filtering instructions 366 direct the system to filter out from further consideration any of the candidate gene markers that comprise a determined effectiveness below a threshold. The candidate gene markers may alternatively be discarded, categorized, and/or flagged for falling below the threshold. The threshold can be any threshold, including a user-defined or user-selected threshold, a system-defined or system-selected threshold, an experimentally derived threshold, and/or any other threshold.

According to an embodiment, comparison instructions 367 direct the system to determine a mean or median expression level of a gene marker that survives filtering, for each of two or more subtypes for which that gene marker is discriminative. The comparison instructions 367 direct the system to compare the mean or median expression level of the gene marker for the first subtype to the mean or median expression level of the same gene marker for the second subtype, to generate an expression level change. This expression level change will either be a positive change in expression or a negative change in expression, depending on the characteristics of the gene marker in the first and second subtypes.

The comparison instructions 367 also direct the system to determine or retrieve from storage a mean or median expression level of the same gene marker from the reference data set for each of two or more subtypes. The comparison instructions 367 direct the system to compare the mean or median expression level of the gene marker for the first subtype to the mean or median expression level of the same gene marker for the second subtype, to generate an expression level change. This expression level change will either be a positive change in expression or a negative change in expression, depending on the characteristics of the gene marker in the first and second subtypes

The comparison instructions 367 also direct the system to compare the first expression level difference obtained from the training data set and the second analytical platform to the second expression level difference obtained from the reference data set and the first analytical platform. If the first expression level difference and the second expression level difference change in the same direction, such as if the first expression level difference is a positive difference and the second expression level difference is also a positive difference, then the candidate gene marker is selected for inclusion in the final list of selected gene markers. Similarly, if the first expression level difference is a negative difference and the second expression level difference is also a negative difference, then the candidate gene marker is selected for inclusion in the final list of selected gene markers.

According to an embodiment, if the first expression level difference and the second expression level difference change in opposite directions, comparison instructions 367 direct the system to select these initially flagged or discarded gene markers for inclusion in the final list of selected gene markers if: (1) the candidate gene marker comprises an expression level above a threshold in the training data set obtained from the second analytical platform; and/or (2) the candidate gene marker comprises an expression level below a threshold in the reference data set obtained from the first analytical platform. This enables recovery of gene markers genotyped via the second analytical platform for inclusion in the final list of selected gene markers even if they are not suitably detectable and/or discriminating when genotyped via the first analytical platform, as long as the gene marker is sufficiently discriminating and survives the filtering steps of the method or system.

The final list of selected gene markers is then presented or otherwise provided to the user via user interface 340. The user interface can be any method, system, or device for conveying, displaying, or transmitting information to a user. For example, the user interface may comprise a screen, a data output or retrieval mechanism, a wired and/or wireless transceiver, or any other device. The user may be a researcher, a physician or other medical professional, or another user developing or using a gene signature.

The methods and systems described or otherwise envisioned herein provide numerous benefits to the gene marker selection system. For example, the gene marker selection system optimizes a gene signature by preferentially selecting a subset of gene markers from a list of candidate gene markers, based on a number of predetermined criteria. Among the possible criteria, the selected gene markers must individually have sufficiently high detectability and discriminative power.

### Example

According to one embodiment, the methods and systems described or otherwise envisioned herein are utilized to preferentially select a subset of gene markers, from a list of candidate gene markers, individually comprising sufficiently high detectability and discriminative power for deciphering between two or more subtypes.

As just one non-limiting implementation of an embodiment of the methods and systems described or otherwise envisioned herein, is an implementation selecting a set of RNA-Seq isoform markers to represent a Wnt-pathway gene signature which was originally designed for single-channel microarray expressions. The implementation utilized TCGA COAD tumor (Wnt active) and normal (Wnt inactive) samples as the training data set for training. The implementation utilized public microarray dataset GSE20916 available on NCBI Gene Expression Omnibus as the reference data set.

As an initial step, the system generates a target-specific decision boundary between the tumor (Wnt active) and normal (Wnt inactive) subtypes. A target-specific decision boundary between two subtypes can be computed from the training data in many ways as described or otherwise envisioned herein. In this Wnt implementation, the boundary was set to be the average of the means of the two subtypes.

A confusion matrix was then generated for the two subtypes and the candidate gene markers in the training data set, using the calculated target-specific decision boundary. The confusion matrix can be generated in many different ways as described or otherwise envisioned herein. In this Wnt implementation, the system implemented a soft counting approach in which it was assumed that all the expressions of a gene marker follow a Gaussian normal distribution centered at the boundary, with the standard deviation computed from the observed measurements. Based on that distribution, the system estimated for each measurement a probability of being associated with one subtype. The entries of the confusion matrix were then identified by summing these probabilities.

Additionally, the system filtered out markers that did not have a sufficiently high expression level, meaning that the gene marker was not sufficiently or reproducibly detectable.

The choice of mean or median expression thresholds k₁, k₂ and k₃ can be adjusted according to the expression distribution of their respective platforms.

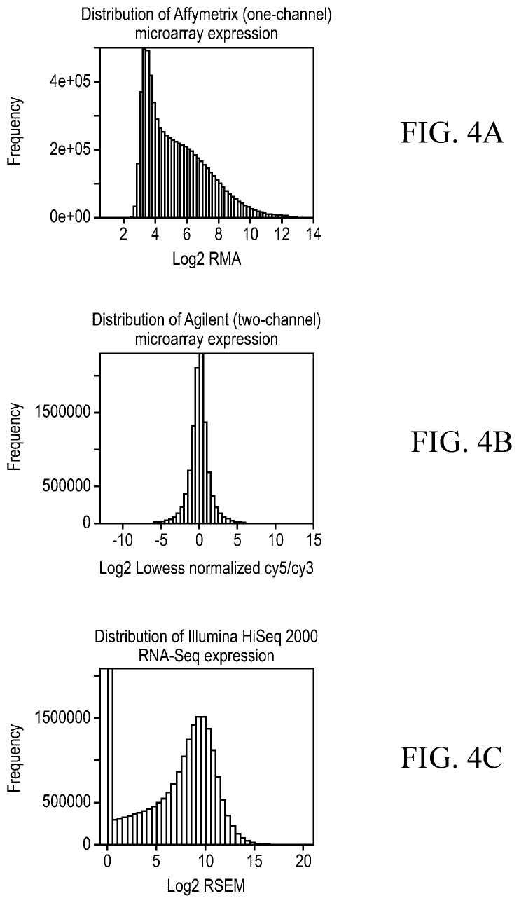

Referring to FIGS. 4A-4B are histogram distributions of the log₂ expressions for Affymetrix (single-channel) microarray platform data from OV studies (FIG. 4A), Agilent (two-channel) microarray platform data from BRCA studies (FIG. 4B), and Illumina HiSeq 2000 RNA-Seq platform data from BRCA studies (FIG. 4C). Since k₂ is the threshold to be applied in case of inconsistent regulation between the primary and alternative platforms, it should be more stringent than k₁, hence k₂>k₁ in general. Accordingly, as just one possible guideline, k₁ and k₃ can be set to the lower 10^{th} percentile and k₂ to the lower 20^{th} percentile, after excluding the undetected expressions from the data distributions of their respective platforms.

In this implementation, the system utilized k₁=2 and k₂=4 for the RNA-Seq platform and k₃=4 for the Affymetrix (single-channel) microarray platform. The approximate values of the 10^{th} to 50^{th} percentile for some common profiling platforms and marker types are listed in Table 1 for reference.

TABLE 1
The 10^{th} to 50^{th} percentiles for the expression
values of six platforms and marker types.

| | Log₂ Expressions | | | | | | |
|---|---|---|---|---|---|---|---|
| Platform and Marker Type | Min. | Max. | 10% | 20% | 30% | 40% | 50% |
| Affymetrix Microarray - Probe | 2.5 | 13.6 | 3.3 | 3.6 | 3.9 | 4.4 | 5.2 |
| Affymetrix Microarray - Gene | 1.7 | 13.4 | 3.3 | 3.5 | 3.9 | 4.4 | 4.9 |
| Agilent Microarray - Probe | −17 | 17 | −1.5 | −0.8 | −0.5 | −0.2 | 0.03 |
| Agilent Microarray - Gene | −11.8 | 14.2 | −1.5 | −0.8 | −0.5 | −0.2 | 0.02 |
| Illumina HiSeq 2000 - Isoform | −7 | 23.1 | 2.8 | 4.4 | 5.5 | 6.5 | 7.5 |
| Illumina HiSeq 2000 - Gene | −8.9 | 21 | 1.6 | 4.2 | 6.1 | 7.4 | 8/3 |

Table provides the minimum and maximum expression values of six platforms and marker types, as well as 10^{th}, 20^{th}, 30^{th}, 40^{th}, and 50^{th} percentiles of those platforms and marker types. The Affymetrix single-channel microarray expressions are in log₂ RMA, the Agilent two-channel expressions in log₂ Lowess normalized cy5/cy3, and the Illumina HiSeq 2000 expressions in log₂ RSEM. The percentiles for the RNA-Seq data are obtained after removing the zero RSEM values.

The system also determined, using the generated confusion matrix, the effectiveness of the candidate gene markers' ability to discriminate between the two subtypes, which was utilized to filter out markers that were not sufficiently discriminating. This filtering can utilize any of the methods described or otherwise envisioned herein. For example, a user or the system can assign different thresholds for different subtypes depending on their goals and preferences. For instance, a user or the system may favor sensitivity over specificity for classification by setting a lower threshold for the former. Alternatively the user or the system may require more accurate classification for certain subtypes by raising the MCC threshold(s).

In this Wnt implementation, the system applied uniform criteria of MCC>0.2 and p-value<0.4. Note that the soft counts from the confusion matrix must be rounded to integers for computing the one-sided hypergeometric p values.

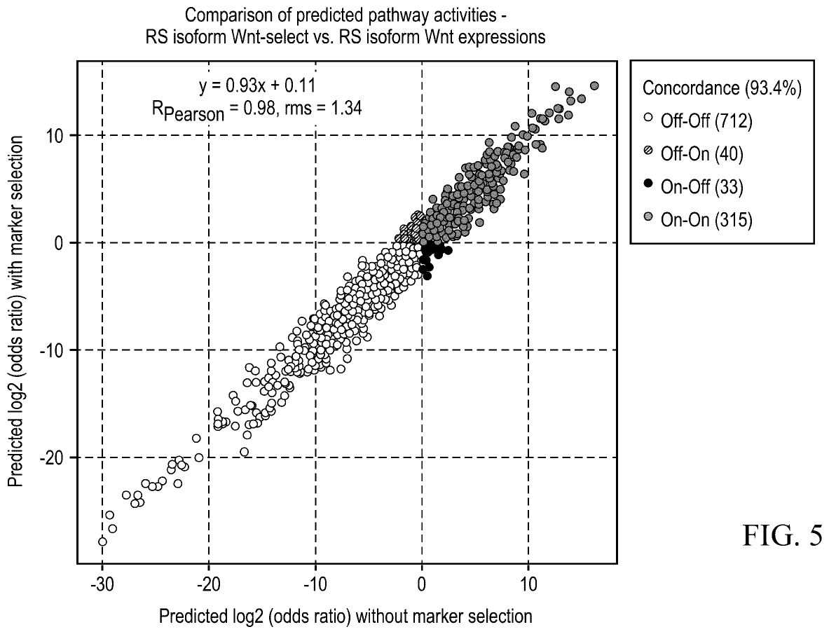

According to an embodiment, this example implementation of the gene marker selection system was used to evaluate the correlation and concordance of the predicted Wnt pathway activities and status with and without marker selection using the TCGA BRCA RNA-Seq version 2 samples. In this non-limiting example, the gene marker selection system and method selected a subset of 69 isoform markers out of a total of 117 markers in the RNA-Seq data, while achieving a high Pearson correlation of 0.98 for the predicted Wnt pathway activities and a high classification concordance of 93.4%, as shown in FIG. 5.

The methods and systems described or otherwise envisioned herein provide numerous benefits to the gene marker selection system. For example, the gene marker selection system optimizes a gene signature by preferentially selecting a subset of gene markers from a list of candidate gene markers, based on a number of predetermined criteria. Among the possible criteria, the selected gene markers must individually have sufficiently high detectability and discriminative power.

As demonstrated, the gene marker selection system and method can adapt a microarray-based signature for RNA-Seq isoform expressions, among other possible adaptations. The gene signature in the example evaluated the Wnt pathway activity and hence predicted its on-off status in a patient based on a Bayesian approach. The gene marker selection system and method resulted in a significant reduction in the number of isoform markers while achieving a high classification concordance compared with the results based on the full signature without marker selection.

According to an embodiment, the gene marker selection system and method can similarly be utilized to identify novel potential gene markers by imposing more stringent selection criteria for all candidate genes. Additionally, other than gene expression, the gene marker selection system and method can also be utilized to select markers of other genomic data, such as methylation, copy number variations, and many more.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for identifying a gene signature comprising one or more selected gene markers each configured to discriminate between two or more tissue or cell subtypes, comprising:
identifying a set of candidate gene markers for discrimination of the two or more subtypes;
providing a reference data set for a first gene expression platform, comprising expression information for a plurality of genes, and further comprising information about a difference in expression of at least some of the plurality of genes between a first subtype and a second subtype;
providing a training data set for a second gene expression platform, comprising expression information for a plurality of genes for each of the first subtype and the second subtype, wherein at least some of the plurality of genes in the training data set are within the set of candidate gene markers;
computing from training data set a boundary gene expression level that distinguishes between the first subtype and the second subtype;
generating, using the computed boundary gene expression level and the expression information for the plurality of genes in the training data set, a confusion matrix for the first subtype and the second subtype;
determining, based on the generated confusion matrix, an effectiveness of each of the plurality of genes in the training data set to discriminate between the first subtype and the second subtype;
filtering from further consideration any of the plurality of genes in the training data set with an expression level below a threshold;
filtering from further consideration any of the plurality of genes in the training data set with a determined effectiveness below a threshold;
calculating a mean and/or median expression level of one of the remaining plurality of genes in the training data set for the first subtype and for the second subtype, and comparing the two calculated mean and/or median expression levels to generate an expression level change for each gene marker between the first subtype and the second subtype;
comparing the generated expression level change for the gene marker to a reference expression level change for the same gene marker in the reference data set; and
selecting the gene marker for inclusion in a list of selected gene markers if the generated expression level change and the reference expression level change are changed in the same direction; and
providing the list of one or more selected gene markers via a user interface, the list comprising the gene signature.

2. The method of claim 1, further comprising:
identifying a gene marker in the training data set for which the generated expression level change and the reference expression level change are changed in opposite directions; and
selecting the gene marker for inclusion in the list of selected gene markers if the gene marker comprises an expression level above a threshold in the training data set, or if the gene marker comprises an expression level below a threshold in the reference data set.

3. The method of claim 2, wherein the threshold is a user-selected threshold.

4. The method of claim 1, wherein the expression level threshold comprises a mean or median expression level obtained from expression information for the plurality of genes in the training data set.

5. The method of claim 1, wherein the effectiveness of each of the plurality of genes in the training data set is determined using one or more of sensitivity, specificity, a Matthew correlation coefficient, and hypergeometric p values.

6. The method of claim 1, wherein the effectiveness of each of the plurality of genes in the training data set is determined using a Matthew correlation coefficient (MCC) and the formula:
$$MCC = \frac{TP \times TN - FP \times FN}{\sqrt{( TP + FP ) \times ( TN + FN ) \times P \times N}}$$
where TP=true positives, FP=false positives, TN=true negatives, FN=false negatives, P=TP+FN, and N=FP+TN.

7. The method of claim 1, wherein the first subtype and the second subtype are cancer subtypes.

8. The method of claim 1, wherein the first gene expression platform is a microarray platform, and the second gene expression platform is an RNA platform.

9. A system for identifying a gene signature comprising one or more selected gene markers each configured to discriminate between two or more tissue or cell subtypes, comprising:
a reference data set comprising expression information obtained from a first gene expression platform for a plurality of genes, and further comprising information about a difference in expression of at least some of the plurality of genes between a first subtype and a second subtype;
a training data set comprising expression information obtained from a second gene expression platform for a plurality of genes, and further comprising information about a difference in expression of at least some of the plurality of genes between a first subtype and a second subtype;
a processor configured to: (i) compute a boundary gene expression level that distinguishes between the first subtype and the second subtype; (ii) generate, using the computed boundary gene expression level and the expression information for the plurality of genes in the training data set, a confusion matrix for the first subtype and the second subtype; (iii) determine, based on the generated confusion matrix, an effectiveness of each of the plurality of genes in the training data set to discriminate between the first subtype and the second subtype; (iv) filter from further consideration any of the plurality of genes in the training data set with an expression level below a threshold; (v) filter from further consideration any of the plurality of genes in the training data set with a determined effectiveness below a threshold; (vi) calculate a mean and/or median expression level of one of the remaining plurality of genes in the training data set for the first subtype and for the second subtype, and comparing the two calculated mean and/or median expression levels to generate an expression level change for each gene marker between the first subtype and the second subtype; (viii) compare the generated expression level change for the gene marker to a reference expression level change for the same gene marker in the reference data set; and (ix) select the gene marker for inclusion in a list of selected gene markers if the generated expression level change and the reference expression level change are changed in the same direction; and
a user interface configured to provide the list of one or more selected gene markers via a user interface, the list comprising the gene signature.

10. The system of claim 9, wherein the processor is further configured to: identify a gene marker in the training data set for which the generated expression level change and the reference expression level change are changed in opposite directions; and select the gene marker for inclusion in the list of selected gene markers if the gene marker comprises an expression level above a threshold in the training data set, or if the gene marker comprises an expression level below a threshold in the reference data set.

11. The system of claim 9, wherein the expression level threshold comprises a mean or median expression level obtained from expression information for the plurality of genes in the training data set.

12. The system of claim 9, wherein the effectiveness of each of the plurality of genes in the training data set is determined using one or more of sensitivity, specificity, a Matthew correlation coefficient, and hypergeometric p values.

13. The system of claim 9, wherein the effectiveness of each of the plurality of genes in the training data set is determined using a Matthew correlation coefficient (MCC) and the formula:
$$MCC = \frac{TP \times TN - FP \times FN}{\sqrt{( TP + FP ) \times ( TN + FN ) \times P \times N}}$$
where TP=true positives, FP=false positives, TN=true negatives, FN=false negatives, P=TP+FN, and N=FP+TN.

14. The system of claim 9, wherein the first subtype and the second subtype are cancer subtypes.

15. The system of claim 9, wherein the first gene expression platform is a microarray platform, and the second gene expression platform is an RNA platform.
